(19) United States
(12) Patent Application Publication
(10) Pub. No.: US 2014/0211151 A1
(43) Pub. Date: Jul. 31, 2014

(54) TORIC LENS CALCULATOR

(71) Applicants: Sanjay Ram Swaroop Argal, Vadodara (IN); Munavvar Tahir Hussain, Vadodara (IN)

(72) Inventors: Sanjay Ram Swaroop Argal, Vadodara (IN); Munavvar Tahir Hussain, Vadodara (IN)

(73) Assignee: Jagrat Natavar DAVE, Vadodara (IN)

(21) Appl. No.: 14/239,277

(22) PCT Filed: Aug. 16, 2012
(86) PCT No.: PCT/IB2012/054177

§ 371 Date: Feb. 18, 2014

(30) Foreign Application Priority Data
Aug. 18, 2011 (IN) 2332/MUM/2011

(51) Int. Cl. A61B 3/00

(52) U.S. Cl. CPC A61B 3/0025
USPC 351/159.74

(57) ABSTRACT

The present invention, in some embodiments thereof, provides a method and device for automatically calculating, prior to an IOL (intraocular lens) implantation procedure in a patient, a power and an axis location of the IOL in the implantation procedure, the method comprising: inputting to an electronic device keratometry parameters of the patient; inputting to the electronic device surgical parameters of the IOL implantation procedure on the patient; analyzing the patient keratometry parameters and the surgical parameters by the electronic device; and automatically determining by the electronic device the power and the axis location of the IOL in the implantation procedure in response to the inputs.

### RELATED APPLICATION

This application claims the benefit of priority of Indian Patent Application No. 2332/MUM/2011 filed Aug. 18, 2011, the contents of which are incorporated herein by reference in their entirety

### FIELD AND BACKGROUND OF THE INVENTION

The present invention relates generally to the field of ophthalmic lenses and, more particularly, to reducing the amount of residual refractive cylinder in patients undergoing IOL (intraocular lens) implantation procedures.

The surgical implantation of an IOL, which replaces an existing natural crystalline lens of the eye, especially one which has been clouded over by a cataract, is well known in the art. The IOL is typically held in place, within the capsular bag inside the eye, by side struts called haptics. This surgery may be performed through a very small incision, thus obviating the need for stitches, in a procedure that may last less than 30 minutes, if performed by an experienced ophthalmologist.

An article by C. K. Patel, S. Ormonde, P. H. Rosen, and A. J Bron entitled “Postoperative Intraocular Lens Rotation: A Randomized Comparison of Plate and Loop Haptic Implants” in Ophthalmology 1999; 106:2190-2196 discusses patients with cataract as the only ocular disease who were randomly implanted with plate or loop haptic implants after uncomplicated phacoemulsification. The baseline position of the IOL was determined from a video frame acquired at the conclusion of surgery. Postoperative IOL position was documented using digital retroillumination images at 2 weeks and 6 months after surgery. Capsular fusion patterns were recorded using slit-lamp biomicroscopy. Correlation of IOL rotation with axial length, capsular contraction, and fusion was attempted.

Such ophthalmic surgery may be performed for the visual correction of aphakia (absence of lens) and pre-existing corneal astigmatism secondary to the removal of a cataractous lens in adult patients with or without presbyopia (loss of lens elasticity), who desire improved uncorrected vision, reduction of residual refractive cylinder, and increased spectacle independence for distance vision. In recent years, cataract and refractive surgery have grown closer. Cataract surgical techniques are now aimed at minimizing aberrations including corneal astigmatism and higher-order defects, and pretreatments for preexisting image defects are commonplace. Very often, cataract procedures also aim to correct myopia (nearsightedness) or hyperopia (farsightedness) by implanting a suitable IOL.

While the replacement lens is most often selected to provide the patient with distance vision, multifocal IOLs are also available, which provide the patient with multiple-focused vision at far and reading distances. Correcting preexisting corneal astigmatism using an astigmatic IOL, to balance out the existing corneal astigmatism that remains postoperatively, and to achieve an overall spherical effect, has also been considered. This may be accomplished with a toric IOL, i.e., an IOL containing at least one toric surface. In addition to providing toric IOLs for the correction of preexisting astigmatic corneal conditions, there may be provided toric IOLs which may additionally correct surgically induced astigmatism (SIA), as discussed herein.

In choosing a toric IOL for a particular patient, a surgeon may consider the amount of the patient's pre-operative corneal astigmatism, generally referred to as keratometry measurements. Additionally, the surgeon may consider the expected amount of surgically induced corneal astigmatism, which may be estimated, depending on the size of the planned incision in the implantation procedure.

The patient's measured pre-operative corneal astigmatism and the estimated amount of surgically induced corneal astigmatism may be used by the surgeon to select a specific toric IOL for the implantation procedure. The particular IOL selected, according to its specific spherical power and cylindrical power, and its axis of placement in the implantation procedure are additional factors to be considered by the surgeon. All of these factors together will have an effect on the post-operative vision of the patient.

European Patent No. 1,732,471, entitled “Method of Calculating the Required Power of a Toric Implant” describes calculating the required power of a toric implant by using the pre-op corneal astigmatism and the predicted surgically induced astigmatism, the latter calculated using power vector analysis of the surgical technique employed by the surgeon. The method uses both the measure pre-operative corneal/ocular induced astigmatism and the predicted surgically-induced astigmatism, the later of which may be predicted using power vector analysis of the surgical technique employed by the surgeon.

“Astigmatism Correction with a Foldable Toric Intraocular Lens in Cataract Patients” by Ruhswurm I, Scholz U, Zehetmayer M, et al. in J. Cataract Refract. Surg. 2000; 26:1022-1027 describes determining the efficacy and rotational stability of a toric posterior chamber silicone IOL to correct preoperative astigmatism in 37 cataract patients. It was determined that mean refractive astigmatism was reduced to 0.84 D; keratometric astigmatism was 2.30 D. In 7 eyes (18.9%), and the IOL axis was rotated a maximum of 25 degrees. In all 37 eyes, the axis of the toric IOL remained within 30 degrees of rotation.

### SUMMARY OF THE INVENTION

The present invention, in some embodiments thereof, provides a method and device for assisting a surgeon in estimating the amount of post-operative corneal astigmatism to be corrected in an IOL (intraocular lens) implantation procedure, in selecting a toric IOL model, and in determining the optimal axis location in the IOL implantation procedure.

The present invention, in some embodiments thereof, provides a method and device which employs pre-operative data in order to calculate an appropriate toric IOL power. Additionally, the present invention, in some embodiments thereof, provides precise calculation of cylindrical power on the corneal plane to get predictable refractive outcomes. The present invention, in some embodiments thereof, additionally provides one or more of a measure of the anticipated residual astigmatism, an optimal axis for IOL placement and the magnitude and axis of anticipated residual astigmatism. Further, the present invention, in some embodiments thereof, provides a wider range of astigmatic correction, for example, from +1 D to +6 D, in 0.5 diopter steps, to accommodate the vast majority of patients with precision.

Accordingly, one objective of the present invention, in some embodiments thereof, is to provide a method for calculating the required power of a toric implant by using both the measured pre-operative corneal/ocular astigmatism and the predicted surgically-induced astigmatism. Still another outcome of the present invention, in some embodiments thereof, is to provide a more accurate method of calculating the required post-operative refractive power of the implant.

This system combines the values of pre-operative corneal astigmatism and surgically induced corneal astigmatism to estimate the post-operative corneal astigmatism. Results of the calculation provide a recommended IOL model, Toric IOL spherical equivalent (SE) power, optimal axis of Toric IOL placement, Toric IOL cylinder (Cyl) power, and Toric IOL Correction (corneal plane).

According to one aspect of the present invention there is provided a method for automatically calculating, prior to an IOL (intraocular lens) implantation procedure in a patient, a power and an axis location of the IOL in the implantation procedure, the method comprising: inputting to an electronic device keratometry parameters of the patient; inputting to the electronic device surgical parameters of the IOL implantation procedure on the patient; analyzing the patient keratometry parameters and the surgical parameters by the electronic device; and automatically determining by the electronic device the power and the axis location of the IOL in the implantation procedure in response to the inputs.

According to some embodiments of the invention, the patient keratometry parameters include: a Flat K measurement of the patient; a Flat Axis degree measurement of the patient; and a Steep K measurement of the patient.

According to some embodiments of the invention, the Flat K measurement and the Steep K measurement may be input in one of diopters and millimeters.

According to some embodiments of the invention, the surgical parameters include: IOL spherical power; SIA (surgically induced astigmatism) measurement; and degree measurement of an incision location for the IOL implantation procedure.

According to some embodiments of the invention, each of the IOL spherical power and SIA measurement may be one of: input in of diopters, if the input Flat K and Steep K are input in diopters; and input in millimeters, if the input Flat K and Steep K are input in millimeters.

According to some embodiments of the invention, the determining comprises determining the optimal power and axis location of the IOL in the implantation procedure.

According to some embodiments of the invention, the electronic device default for the SIA parameter is about 0.50 diopters.

According to some embodiments of the invention, the method further comprises outputting the power and the axis location of the IOL in the implantation procedure by the electronic device.

According to some embodiments of the invention, the outputting comprises outputting the following: at least one suggested toric IOL model for use in the IOL implantation procedure; and an orientation for each of the at least one suggested toric IOL models.

According to some embodiments of the invention, the method further comprises outputting at least one of: a pre-op corneal astigmatism calculation; a crossed-cylinder result (corneal plane) calculation; and an anticipated residual astigmatism after the IOL implantation procedure, for each at least one suggested toric IOL models.

According to some embodiments of the invention, the at least one suggested toric IOL model includes two suggested toric IOL models.

According to some embodiments of the invention, the outputting includes outputting an eye diagram including an orientation for each of the at least one suggested toric IOL models.

According to some embodiments of the invention, the outputting includes providing an eye diagram with reference axis markings, the eye diagram indicating an incision location for and an axis location of the IOL in the implantation procedure.

According to some embodiments of the invention, the outputting is selected from displaying the power and the axis location of the IOL in the implantation procedure on a display and transmitting the power and the axis location of the IOL in the implantation procedure to a printing device.

According to some embodiments of the invention, the automatically determining includes: calculating the pre-op corneal astigmatism from the difference between an input Flat K measurement and an input Steep K measurement; calculating the crossed-cylinder result (corneal plane) from a combination of the calculated pre-op corneal astigmatism and an input surgically induced astigmatism (SIA) measurement; and calculating the anticipated residual astigmatism from the difference between the calculated crossed cylinder result (corneal plane) and a cylinder power correction (corneal plane) of a suggested IOL model.

According to some embodiments of the invention, the suggested IOL model is acquired from a database of toric IOL data.

According to some embodiments of the invention, the cylinder power correction of the suggested IOL model is acquired from a database of toric IOL data.

According to another aspect of the present invention, there is provided a system for automatically calculating, prior to an IOL (intraocular lens) implantation procedure in a patient, the power and axis location of the IOL in the implantation procedure, the system comprising: an input device configured for receiving keratometry parameters of the patient and surgical parameters of the IOL implantation procedure on the patient; a processor configured for analyzing the patient keratometry parameters and the surgical parameters received from the input device and automatically calculating the power and axis location of the IOL in the implantation procedure; and a memory device configured for storing data.

According to some embodiments of the invention, the patient keratometry parameters include: a Flat K measurement of the patient; a Flat Axis degree measurement of the patient; and a Steep K measurement of the patient.

According to some embodiments of the invention, the Flat K measurement and the Steep K measurement may be input in one of diopters and millimeters.

According to some embodiments of the invention, the surgical parameters include: IOL spherical power; SIA (surgically induced astigmatism) measurement; and degree measurement of an incision location for the IOL implantation procedure.

According to some embodiments of the invention, each of the IOL spherical power and SIA measurement may be one of: input in of diopters, if the input Flat K and Steep K are input in diopters; and input in millimeters, if the input Flat K and Steep K are input in millimeters.

According to some embodiments of the invention, the processor is configured for automatically calculating the optimal power and axis location of the IOL in the implantation procedure.

According to some embodiments of the invention, the electronic device default for the SIA parameter is about 0.50 diopters.

According to some embodiments of the invention, the system further comprises an output device configured for outputting the power and axis location of the IOL in the implantation procedure.

According to some embodiments of the invention, the output device is configured for outputting the following: at least one suggested toric IOL model; and an orientation for each of the at least one suggested toric IOL models.

According to some embodiments of the invention, the output device is configured for outputting at least one of: a pre-op corneal astigmatism calculation; a crossed-cylinder result (corneal plane) calculation; and an anticipated residual astigmatism after the IOL implantation procedure, for each at least one suggested toric IOL models.

According to some embodiments of the invention, the at least one suggested toric IOL model includes two suggested toric IOL models.

According to some embodiments of the invention, the output device is configured for outputting an eye diagram including an orientation for each of the at least one suggested toric IOL models.

According to some embodiments of the invention, the output device is configured for outputting an eye diagram with reference axis markings, the eye diagram indicating an incision location for and an axis location of the IOL in the implantation procedure.

According to some embodiments of the invention, the output device is selected from a display device and a printing device.

According to some embodiments of the invention, the pre-op corneal astigmatism is calculated from a difference between an input Flat K measurement and an input Steep K measurement; wherein the crossed-cylinder result (corneal plane) is calculated from a combination of the calculated pre-op corneal astigmatism and an input surgically induced astigmatism (SIA) measurement; and wherein the anticipated residual astigmatism is calculated from the difference between the calculated crossed cylinder result (corneal plane) and a cylinder power correction (corneal plane) of a suggested IOL model.

According to some embodiments of the invention, the suggested IOL model is acquired from a database of toric IOL data.

According to some embodiments of the invention, the cylinder power correction of the suggested IOL model is acquired from a database of toric IOL data.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, in some embodiments thereof, suitable methods and materials are described below. In case of conflict, the patent specification, including definitions, will control. In addition, the materials, methods, and examples are illustrative only and not intended to be limiting.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of.”

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of ophthalmology.

Implementation of the method and system of the present invention, in some embodiments thereof, involves performing or completing selected tasks or steps manually, automatically, or a combination thereof. Moreover, according to actual instrumentation and equipment of preferred embodiments of the method and system of the present invention, in some embodiments thereof, several selected steps could be implemented by hardware or by software on any operating system of any firmware or a combination thereof. For example, as hardware, selected steps of the invention, in some embodiments thereof, could be implemented as a chip or a circuit. As software, selected steps of the invention, in some embodiments thereof, could be implemented as a plurality of software instructions being executed by a computer using any suitable operating system. In any case, selected steps of the method and system of the invention, in some embodiments thereof, could be described as being performed by a data processor, such as a computing platform for executing a plurality of instructions.

### BRIEF DESCRIPTION OF THE DRAWINGS

The invention, in some embodiments thereof, is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention, in some embodiments thereof, may be embodied in practice.

In the drawings:

FIG. 1 is a block diagram of the system, in accordance with some embodiments of the present invention;

FIG. 2 is a flowchart of a method in accordance with some embodiments of the present invention;

FIG. 3 is a schematic illustration showing the inputting of information in accordance with the method of some embodiments of the present invention;

FIG. 4 is a schematic illustration showing the outputting of information in accordance with the method of some embodiments of the present invention;

FIG. 5 illustrates an exemplary input screen in accordance some embodiments of the system of the present invention;

FIG. 6 illustrates an exemplary output screen in accordance with some embodiments of the system of the present invention; and

FIG. 7 is chart illustrating the cylinder power and range of anticipated residual astigmatism for a variety of toric IOL models, in accordance with some embodiments of the present invention.

### DESCRIPTION OF THE PREFERRED EMBODIMENTS

The present invention, in some embodiments thereof, is a method and device for automatically calculating, prior to an IOL implantation procedure in a patient, a power and an axis location of the IOL in the implantation procedure, Specifically, the present invention, in some embodiments thereof, can be used to provide an ophthalmological surgeon with the optimum power and axis location of an IOL to be used in an implantation procedure for a particular patient. Additionally, the present invention, in some embodiments thereof, provides outputting at least one suggested toric IOL model for use in the IOL implantation procedure and an orientation within the eye for each of the at least one suggested toric IOL models.

The principles and operation of the method and device according to the present invention, in some embodiments thereof, may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

As discussed herein, the present invention, in some embodiments thereof, includes calculating the anticipated residual astigmatism resulting from an IOL implantation procedure, taking into account patient factors and surgeon factors. Patient factors include all of surgically induced astigmatism (SIA) of the implant, the K-reading for the steepest meridian and axis of the patient and the K-reading for the flattest meridian and axis if the patient, incision location, and the IOL spherical power. Surgeon factors include size and location of the incision and the surgically induced refractive change, or surgically induced astigmatism (SIA) typical for the individual surgeon. The cylinder axis in clinical prescription usually falls between 0 and 180 degrees. The current corneal incision procedure of cataract surgery causes both the flattening and the steepening of the corneal surface at meridians associated with the incision locations. This creates a measurable cylinder power change and cylindrical axis shift in post-operative refraction. Surgically induced astigmatic change should be taken into account in predicting the post-operative astigmatism and then it is possible to use a toric implant to neutralize the astigmatism in the whole eye. As will be discussed herein, the postoperative corneal astigmatism orientation and incision location may be displayed on a schematic diagram of the eye located on an output display screen. The recommended IOL orientation may be displayed as well.

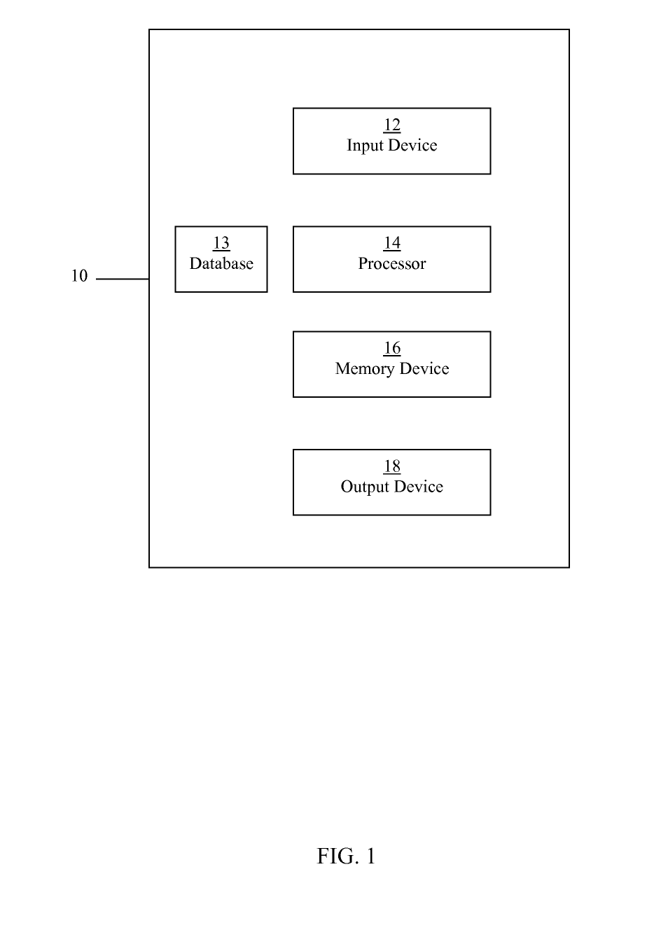

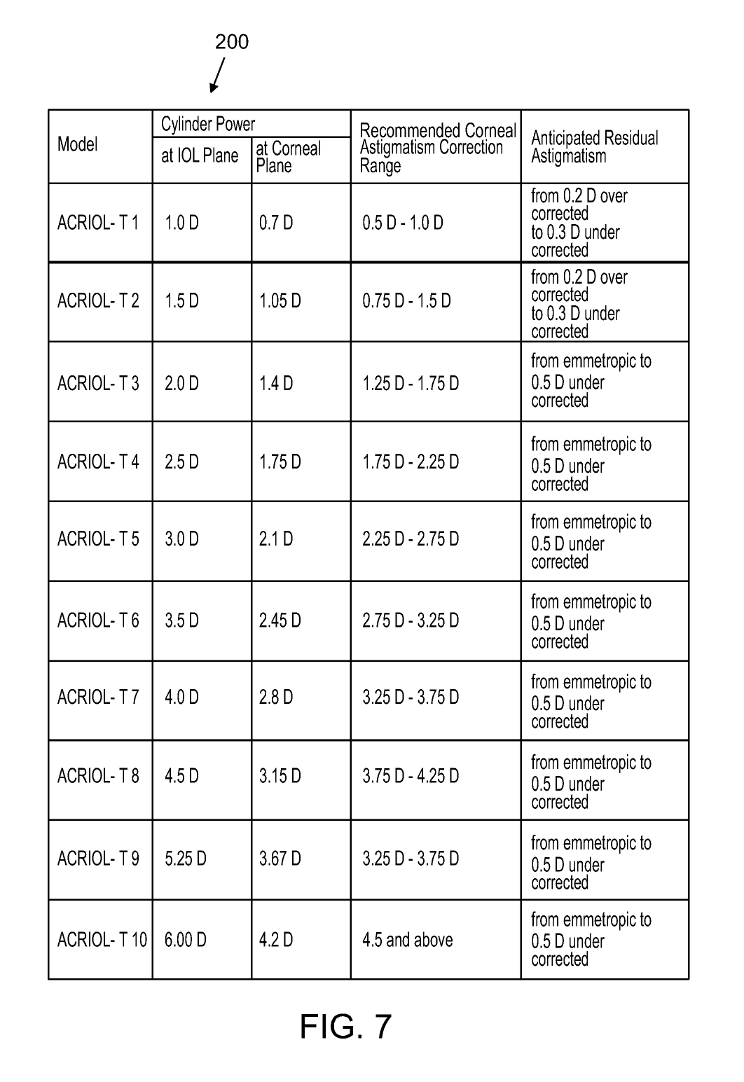

Referring now to the drawings, FIG. 1 is a block diagram of the system, in accordance with some embodiments of the present invention. The system 10 includes an input device 12 which may be, for example, a keyboard or mouse, via which a user may input keratometry parameters of a patient and surgical parameters of a selected IOL implantation procedure on the patient. System 10 additionally includes a processor 14 for analyzing the keratometry parameters and the surgical parameters received from the input device 12 and for automatically calculating the power and axis location of an IOL to be employed in the implantation procedure. System 10 further includes a memory device 16 for storing data, which includes both data input via input device 12 and data calculated by processor 14. Further, system 10 preferably includes an output device 18 such as, for example, an output display or printer for outputting data, as discussed herein. The system 10 automatically calculates, prior to an IOL (intraocular lens) implantation procedure in a patient, the power and axis location of the IOL in the implantation procedure, as discussed herein. The system may use data acquired from a database 13, as discussed further herein, in particular with reference to FIG. 7. The system may also output at least one suggested toric IOL model and an orientation for each of the at least one suggested toric IOL models, as described in detail herein.

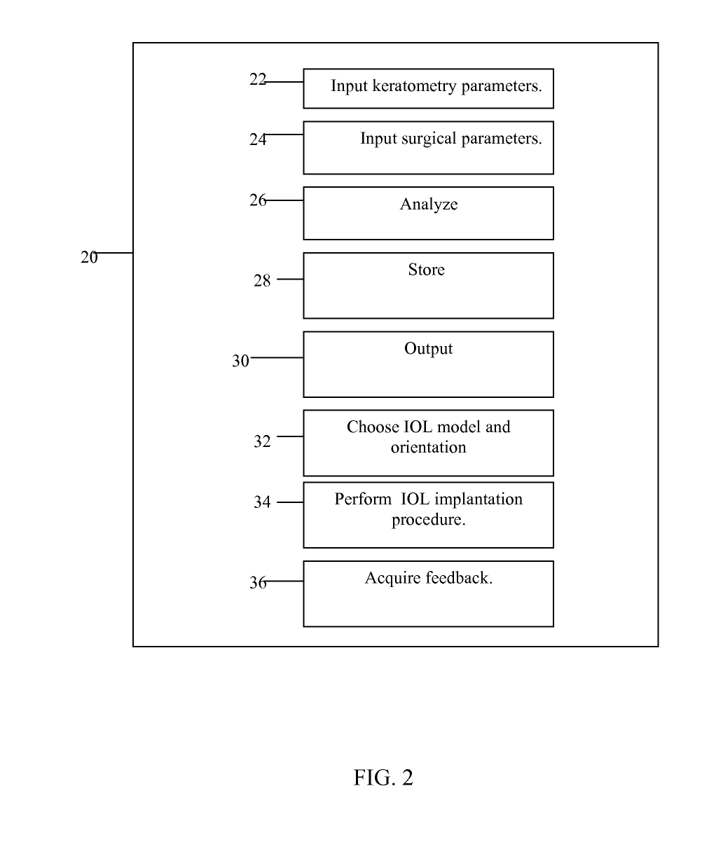

FIG. 2 is a flowchart of a method in accordance with some embodiments of the present invention. Method 20 includes inputting, at 22, keratometry parameters of a patient. These keratometry parameters include a Flat K measurement of the patient, a Flat Axis degree measurement of the patient, and a Steep K measurement of the patient. These keratometry parameters may be input in one of diopters and millimeters, as discussed further herein.

Method 20 additionally includes inputting, shown at 24, surgical parameters of an IOL implantation procedure on the patient. These surgical parameters include the IOL spherical power, the SIA (surgically induced astigmatism) measurement, and the degree measurement of an incision location for the IOL implantation procedure.

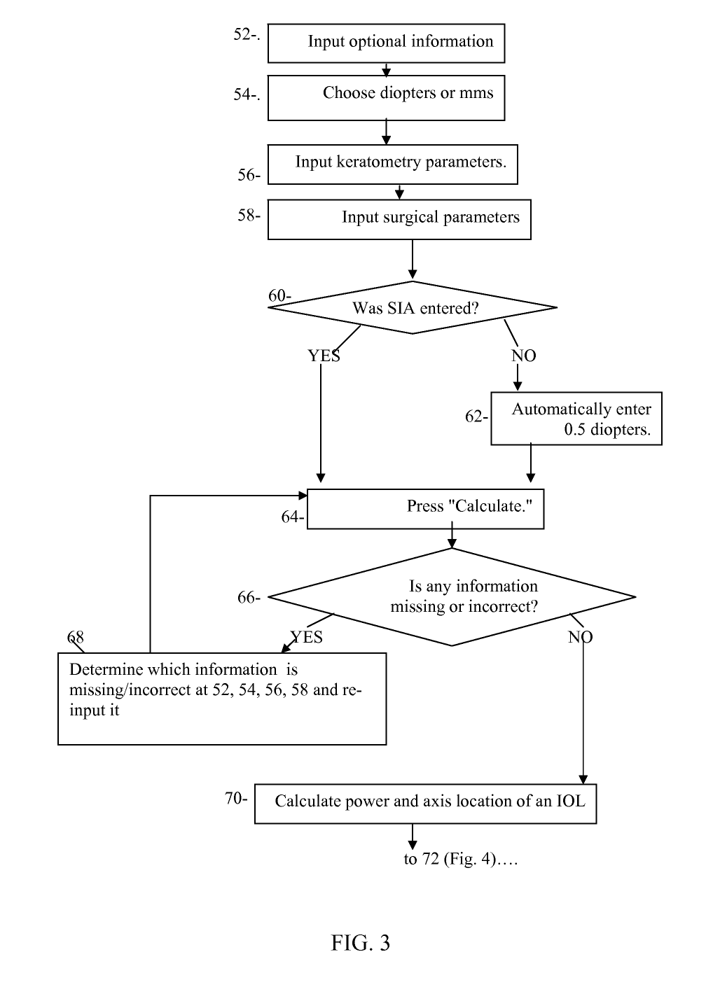

Inputting at 22 and inputting at 24 are discussed herein in further detail, with reference to FIG. 3.

Method 20 additionally includes analyzing, at 26, the patient keratometry parameters input at 22 and the surgical parameters input at 24, such that the power and the axis location of the IOL in the implantation procedure in response to those inputs is automatically calculated, prior to an IOL implantation procedure in a patient. Preferably, in accordance with some embodiments of the invention, the calculated power and axis location are the optimal power and axis location of the IOL in the implantation procedure.

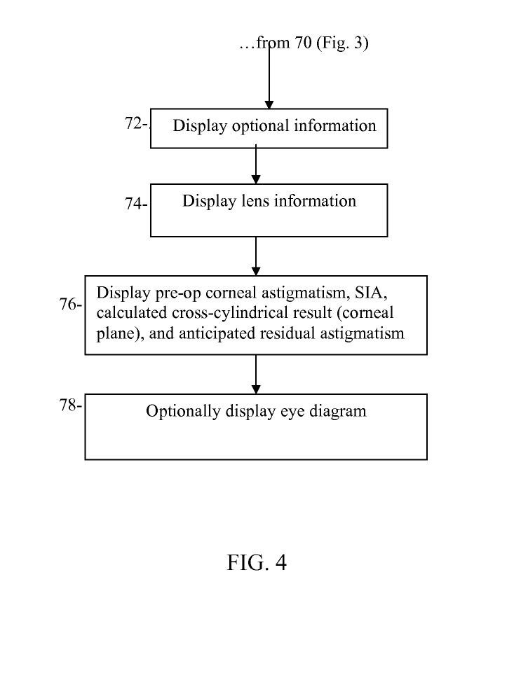

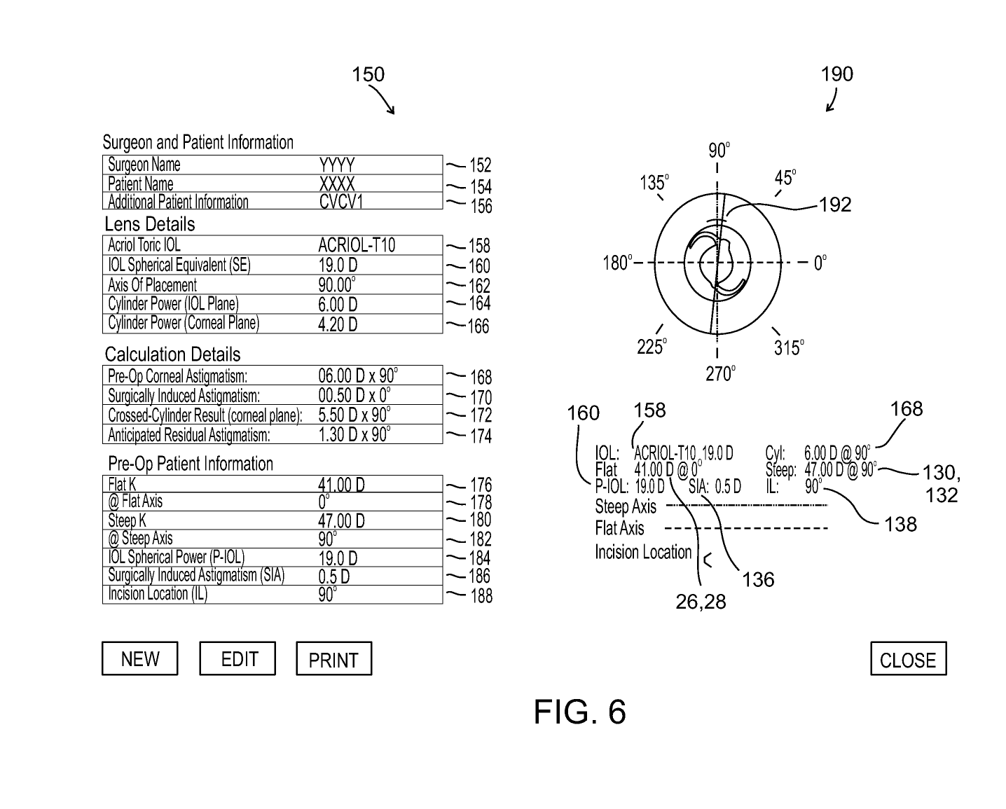

Method 20 further includes storing, at 28, the keratometry parameters input at 22 and the surgical parameters input at 24, as discussed further herein, in particular with reference to FIGS. 4 and 6. Storing 28 additionally includes storing the power and axis location calculated at 26. Optionally, storing 28 may include storing additional calculated data, as described herein with reference to FIGS. 4 and 6.

Method 20 further includes outputting, at 30, the power and the axis location of the IOL in the implantation procedure. Optionally, at least one of the following may additionally be output: at least one suggested toric IOL model for use in the IOL implantation procedure, and an orientation for each of the at least one suggested toric IOL models. Preferably, the at least one suggested toric IOL model includes two suggested toric IOL models.

Optionally, outputting 30 additionally includes outputting at least one of: a pre-op corneal astigmatism calculation, a crossed-cylinder result (corneal plane) calculation, and an anticipated residual astigmatism after the IOL implantation procedure, for each at least one suggested toric IOL models.

Optionally, outputting 30 additionally includes outputting an eye diagram including an orientation for each of the at least one suggested toric IOL models. Preferably, such an eye diagram includes reference axis markings, the eye diagram indicating an incision location for and an axis location of the IOL in the implantation procedure.

Preferably, outputting 30 includes one of displaying the power and the axis location of the IOL in the implantation procedure on a display and transmitting the power and the axis location of the IOL in the implantation procedure to a printing device.

Preferably, method 20 further includes, at 32, choosing an IOL model and orientation from those output at 30; at 34, performing an IOL implantation procedure using the selected IOL model at the suggested orientation; and, after the surgery 34, acquiring at 36 feedback regarding the IOL implantation surgical procedure for the surgeon. This feedback may be acquired by, for example, testing the near and distance post-operative vision of the patient and comparing with expected results had the method 20 in accordance with the present invention not been employed, to determine whether the invention provides a wider range of astigmatic correction.

FIG. 3 is a schematic illustration showing the inputting of information in accordance with some embodiments of the method of the present invention. As shown at 52, a user may optionally input information relating to the surgeon and the patient, for example, at least one of surgeon's name, patient's name, and additional patient information. Other optional information may be input, if desired such as, for example, the patient's age and/or date of birth, and which eye (right or left) is to have IOL implantation procedure performed on it. This information may be used by the surgeon for reference.

As noted above with reference to FIGS. 1-2, in accordance with some embodiments of the present invention, the input information and output data may be stored. Optionally, the stored information may be stored as either patient-dependent or physician-dependent. For example, the system may keep track of what actions are performed by the surgeon and compare these with a suggested procedure such as, for example, a suggested toric IOL model and axis of orientation, as discussed herein. Additionally, certain patient data regarding an implantation procedure may provide information which may be utilized select an implant to be used in a future implantation procedure, or even to predict the outcome of a future implantation procedure such as, for example, performed on the patient's other eye. In this manner, analysis and calculations may be fine-tuned for future procedures.

At 54, the user inputs the units of measurement, i.e., one of diopters or millimeters, that will be used when entering many of the remaining inputs, for example at inputs 56, 58, 70, 72, and 78 below. While the user must choose the units of measurement, he may choose either diopters or millimeters, according to his preference.

Keratometry parameters of the patient are required to be input at 56, these parameters including all of a Flat K measurement of the patient, a Flat Axis degree measurement of the patient, and a Steep K measurement of the patient. The first and third of these keratometry measurements must be input in one of diopters or millimeters, depending on the choice of unit input at 54. If the unit chosen at 54 was diopters, then the acceptable range for each of these parameters is from 30.0 to 60.0 D (diopters), which should cover up to 99.9% of patients undergoing an IOL implantation procedure; if the unit chosen was millimeters, then the acceptable range is from 11.25 to 5.63 mm. It may be noted that a preferred range for these parameters is from 35.0-50.0 D (9.64-6.75 mm), which should cover up to 95% of patients.

As shown at 58, the user then inputs surgical parameters, which include IOL spherical power, an SIA (surgically induced astigmatism) measurement, and a degree measurement of an incision location for the IOL implantation procedure. If the unit chosen at 54 was diopters, then the acceptable ranges for these parameters are from 0.0-40.0 diopters, 0.00-2.0 diopters, and 0-360 degrees, respectively. If the unit chosen was millimeters, then the acceptable range for the IOL spherical power is from 0.0-8.43 mm; the SIA must be input in diopters. It may be noted that a preferred range for the IOL spherical power is from 6.0-30.0 D (56.25-11.25 mm), which are the spherical powers of the most commonly used IOLs.

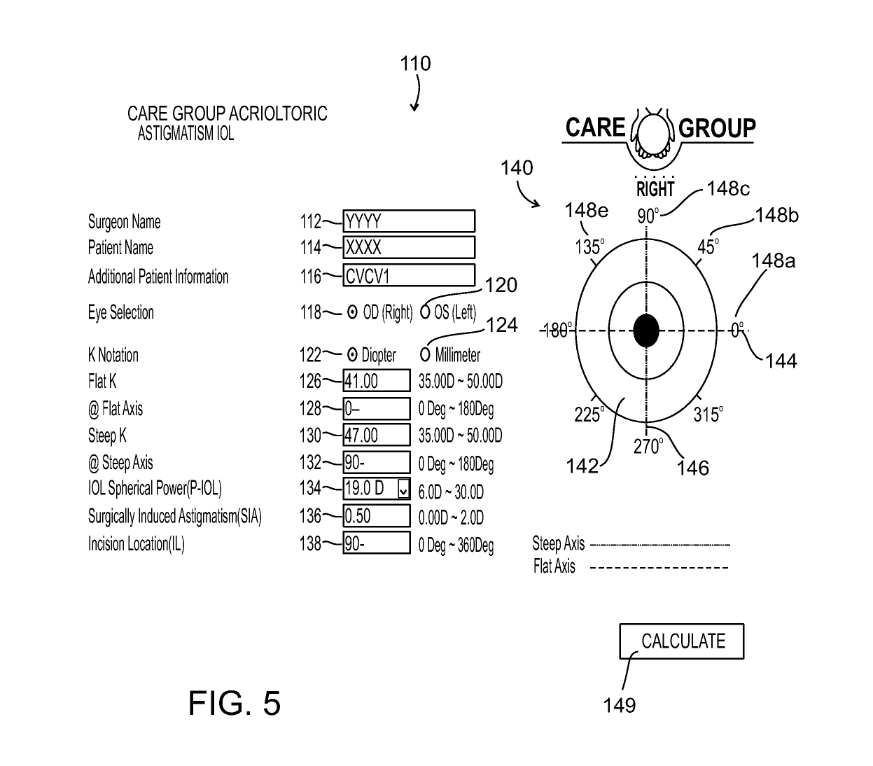

At 60, it is determined whether or not an SIA value was entered by the user. If it was entered, the method proceeds towards 64, but if it was not entered by the user, a value of 0.5 diopters is automatically input and then the method proceeds towards 64. At 64, the user initiates a calculating procedure, wherein the input data is processed, as discussed herein with regard to FIGS. 5-6.

At 66, it is determined whether any of the data that was input at 54, 56, and 58 is incomplete or is outside a required range. If this is so, then the user is prompted, at 68, for example by an error message conveyed to him, to reenter the missing or incorrect data. The user must then reinput the necessary information and click “Calculate” again, after which the method proceeds toward 70. Alternatively, if at 66 it is determined that each item of data that was input at 54, 56, and 58 is indeed complete and within its required range, then the method proceeds toward 70. In accordance with the method 10, at 70 a power and an axis location of an IOL is automatically calculated, as discussed further herein.

FIG. 4 is a schematic illustration showing the outputting of information in accordance with some embodiments of the method of the present invention. After the user has performed the portion of the method in accordance with the procedure shown in FIG. 3, the method continues as shown in FIG. 4. At 72, information relating to the surgeon and the patient, specifically that which was input at 52 (FIG. 3) may be displayed.

At 74, the power and the axis location of the IOL in the implantation procedure may be displayed. This information may include at least one suggested toric IOL model and, for each model, a spherical power, a suggested axis of orientation, a cylindrical power (IOL plane) and a cylindrical power (corneal plane). Preferably, the method calculates the optimal power and axis location of the IOL to be used in the implantation procedure. Optionally, at least two suggested toric IOL models are displayed, together with the relevant data noted herein.

Further in accordance with some embodiments of the method in accordance with the present invention, at 76, for each at least one suggested toric IOL models, at least one of a pre-op corneal astigmatism calculation, a crossed-cylinder result (corneal plane) calculation, and an anticipated residual astigmatism after the IOL implantation procedure, is displayed.

At 78, for each of the at least one suggested toric IOL models, an eye diagram may be displayed, as will be discussed herein, the eye diagram including an orientation, reference axis markings, and an incision location for and an axis location of the IOL in the implantation procedure.

It is to be understood that, instead of displaying the information on a display, as discussed herein with regard to FIG. 4, the data may be transmitted to a printing device such that a hard copy of the output information may be obtained.

FIG. 5 illustrates an exemplary input screen in accordance with some embodiments of the present invention. Input screen 110 includes various fields in which a user such as, for example, an ophthalmologist who is to perform an IOL implantation procedure, may input information relating to a particular patient, prior to the IOL implantation procedure, in order to calculate an optimum power and axis location of the IOL to be used in the implantation procedure. As shown in the drawing, there are optionally provided fields 112, 114, and 116 for optionally inputting the surgeon's name, the patient's name, and additional patient information, respectively, as discussed above. These fields may be preceded by words or phrases indicating the type of information requested, for example, “surgeon's name,” “patient's name,” and “additional patient information,” respectively. Additionally, if desired, any of the fields may be grouped together on the input screen, and may optionally include headings, so as to make the input screen more user-friendly. For example, surgeon and patient information may be grouped together under the heading “surgeon and patient information.” Similarly, output information, as will be discussed herein with reference to FIGS. 6, may be grouped together, for example, under headings such as, for example, “lens details,” “calculation details,” and “pre-op patient information.”

A pair of check boxes 118 and 120 may be provided, so that the user may indicate which eye, the right or left, is to have the IOL implantation procedure performed on it. Only one of these check boxes 118 and 120 may be ticked.

It is known in the art for an ophthalmologist to use a keratometer, also known as an ophthalmometer, which is a diagnostic instrument for measuring the curvature of the anterior surface of the cornea, particularly for assessing the extent and axis of astigmatism. These measurements include a Flat K measurement; a Flat Axis degree measurement, and a Steep K measurement.

In accordance with the invention, in some embodiments thereof, a user may select whether to input the patient's keratometry measurements in one of diopters or millimeters, by ticking one of the check boxes 122 and 124, respectively. The user may then input the patient's Flat K measurement, flat axis angle, and Steep K measurement, in fields 126, 128, and 130, respectively. The flat axis angle entered in field 128 must be in the range of from 0-180 degrees. The measurements input in fields 126 and 130 must be input in accordance with the units selected in either of boxes 122 and 124, noted herein. It should be noted that, if the user chooses diopters, by ticking check box 122, each of the Flat K measurement input in field 126 and the Steep K measurement input in field 130 must be input in diopters. Additionally, when input in diopters, the Flat K and Steep K measurements input in respective fields 126 and 130 must each be in the range of from 30.0-60.0 diopters, as noted above. Further, when input in diopters, the Flat K measurement must be less than or equal to the Steep K measurement.

Alternatively, if the user chooses millimeters, by ticking check box 124, each of the Flat K measurement input in field 126 and the Steep K measurement input in field 130 must be input in millimeters. Additionally, when input in millimeters, the Flat K and Steep K measurements input in respective fields 126 and 130 must each be in the range of from 6.75-9.64 mm. Further, when input in millimeters, the Flat K measurement must be greater than or equal to the Steep K measurement.

It may be noted that, once the user has input the Flat K Axis in field 128, the Steep K Axis, the meridian of the steepest corneal power, will be automatically calculated, as known in the art, based on the Flat K axis value, and will be automatically displayed in degrees in a field 132.

In accordance with the present invention, in some embodiments thereof, the input screen further includes fields 134, 136, and 138 in which a user may input surgical parameters of the IOL implantation procedure, these surgical parameters including an IOL spherical power, the surgeon's estimated surgically induced astigmatism (SIA), and the incision location, respectively. These fields 134, 136, and 138 may accept inputs in the ranges of 6.0-30.0 diopters, 0.00-2.0 diopters, and 0-360 degrees, respectively.

It should be noted that, if the user chose diopters, by ticking check box 122, each of the IOL spherical power, input in field 134, and the surgeon's estimated surgically induced astigmatism (SIA), input in field 136, must be input in diopters. Additionally, when input in diopters, the IOL spherical power and SIA inputs in respective fields 134 and 136 must be in the range of from 0.0-40.0 diopters and 0.00-2.0 diopters, respectively. Alternatively, if the user chose millimeters, by ticking check box 124, the input in field 134 must be input in millimeters; field 136 must be input in diopters only. Additionally, when input in millimeters, the input for field 134 must be in the range of from 0.0-8.43 mm.

Optionally, a user may not enter any input in field 136, for example, if he does not know how much astigmatism his incision will induce. In this case, the device will automatically analyze the data using a default input of 0.50 diopters for the SIA parameter.

In accordance with the invention, in some embodiments thereof, input screen 110 optionally includes a reference diagram 140 illustrating an eye 142 having a horizontal (Flat) reference axis 144, shown at 0 and 180 degrees, and a vertical (Steep) reference axis 146, shown at 190 and 270 degrees. Reference diagram 140 may be additionally provided with indications of angular measurements relative to the eye 142 such as, for example, angles 0, 45, 90, and 135, shown at ref. nos. 148a-d, respectively.

The input screen may include an active area 149, for example, a rectangle displaying the word “Calculate” which may be triggered, for example, by clicking with a mouse. Such triggering causes the system to automatically calculate a power and an axis location of an IOL, as discussed herein.

If any information that was entered in the input fields discussed herein is incomplete or outside required ranges, then an error message will be displayed on the input screen, indicating the field(s) in which incomplete or inappropriate information was entered. The user is thus prompted to reinput the necessary information and click “Calculate” again.

In accordance with the present invention, in some embodiments thereof, there is provided a processor (FIG. 1) which analyzes the patient keratometry parameters discussed herein, input in fields 126, 128, and 130 and the surgical parameters, also discussed herein, input in fields 134, 136, and 138. The system automatically calculates a power and an axis location of an IOL in the implantation procedure in response to analysis of these inputs. Preferably the system calculates the optimal power and axis location of an IOL to be used in the implantation procedure.

FIG. 6 is an exemplary output device in accordance with some embodiments of the present invention. There is shown an output screen 150, wherein various information may be displayed, as discussed herein. Output screen 150 optionally displays surgeon and patient information, for example, surgeon's name, patient's name, and additional patient information, in fields 152, 154, and 156, respectively. These fields correspond to surgeon and patient information optionally input in fields 112, 114, and 116 (FIG. 5), respectively, discussed herein.

Based on the analysis of data input in input screen 110 and reference to a database of toric IOL data, discussed further herein with reference to FIG. 7, output screen 150 additionally may display lens details which may include the following: a suggested type of toric IOL model 158, for example, “Acrol-T10” to be used in the implantation procedure; the spherical power 160 of the suggested toric IOL model; a suggested axis of orientation 162 for the suggested toric IOL model; a cylinder power (IOL plane) 164; and a cylinder power (corneal plane) 166. It may be noted that, generally, the axis of orientation 162 will be the same as the Steep axis input at 132 (FIG. 5), discussed above. In accordance with the present invention, in some embodiments thereof, the suggested toric IOL model will represent an option which will minimize the residual cylinder.

It may be noted that, if the check box 122 corresponding to diopters was ticked input screen 110 (FIG. 5), then the lens details and pre-op patient information will be displayed in diopters. Alternatively, if the check box check box 124 corresponding to millimeters was ticked input screen 110 (FIG. 5), then the lens details and pre-op patient information will be displayed in millimeters.

Optionally, if check box 122 was ticked, such that “diopters” was chosen as the input measurement, the lens details and pre-op patient information may be displayed in millimeters. Conversely, if check box 124 was ticked, such that “millimeters” was chosen as the input measurement, the lens details and pre-op patient information may optionally be displayed in diopters. Alternatively, equivalent diopter and millimeter measurements may be displayed, regardless of which check box (122 or 124) was ticked.

Additionally, the output screen 150 may display calculation details such as, for example, pre-op corneal astigmatism calculation 168, a surgically induced astigmatism measurement 170, a calculated crossed-cylinder result (corneal plane) 172, and a calculated anticipated residual astigmatism 174. It may be noted that the surgically induced astigmatism measurement 170 is the same as that input in field 136 (FIG. 5), discussed herein.

It may be noted that the pre-op corneal astigmatism is calculated from the difference between the input Flat K measurement and input Steep K measurement, as known in the art. It may additionally be noted that the crossed-cylinder result (corneal plane) is calculated from the difference between the pre-op corneal astigmatism and surgically induced astigmatism at the corneal plane, discussed further herein, with reference to FIG. 7. It may further be noted that the anticipated residual astigmatism is calculated from the difference between the calculated crossed cylinder result (corneal plane) and a cylinder power correction (corneal plane) of a toric IOL model. Data for the toric IOL model is determined from a database of toric IOL data, as discussed further herein with reference to FIG. 7.

The output screen may additionally display pre-op patient information such as, for example, the patient keratometry measurements including the Flat K measurement 176, Flat axis angle 178, Steep K measurement 180, Steep axis angle 182, IOL spherical power 184, SIA 186, and angle of incision location 188. It may be noted that the measurements 176, 178, 180, and 182 displayed will be identical to those input by the user in input fields 126, 128, 130, and 132, respectively. Also, the output IOL spherical power 184, SIA 186, and incision location 188 displayed will be identical to those input by the user in input fields 134, 136, and 138, respectively.

In accordance with the discussion herein with regard to FIG. 5, the measurements displayed in fields 176, 180, 184, and 186 will be in one of diopters and millimeters, corresponding to the user's choice of one of diopters and millimeters, which depends on which one of check boxes 122 and 124 (FIG. 5) were ticked previously.

Optionally, output screen 150 may include at least one additional suggested type of toric IOL model (not shown) and relevant information, such as that discussed herein and displayed for toric IOL model 158. This additional suggested type of toric IOL model will represent the closest available cylinder power(s) for comparison. If desired, output screen 150 may include two or more additional suggested types of toric IOL models.

In accordance with the invention, in some embodiments thereof, output screen 150 optionally includes, for each suggested toric IOL model, a reference eye diagram 190, of which one is shown, which includes all the information and reference indications shown in reference diagram 140 (FIG. 5). On each reference eye diagram 190, there is additionally shown an indication, for example, an arc 192, of an optimal orientation for a suggested toric IOL model. In diagram 190 shown in FIG. 6, the optimal orientation is shown at 90 degrees.

By providing a user, such as a surgeon, with a plurality of suggested IOL model types and cylinder powers, he may choose any of the suggested implants, depending on the anticipated residual astigmatism and desired result. If the anticipated residual astigmatism of one of the models is too low or too high, he may choose to implant one of the other suggested models.

If desired, instead of displaying the input and calculated information on a display, this information may be transmitted to a printing device for printing, as known in the art.

### EXAMPLES

Reference is now made to the following examples which, together with the descriptions herein, illustrate the invention, in some embodiments thereof, in a non limiting fashion.

FIG. 7 shows suggested IOL models and relevant data for each model. The data may is shown in a chart 200 and may form part of a database which to be used in calculations in accordance with embodiments of the present invention, discussed further herein. The data include the cylinder power and range of anticipated residual astigmatism for a variety of toric IOL models. For example, for an Acriol-T1, having a cylinder power of 1.0 diopter in the IOL plane and 0.7 diopters at the corneal plane, the anticipated residual astigmatism is from 0.2 diopters overcorrected to 0.3 diopters undercorrected.

In another example, for which output data are shown in FIG. 6, in order to provide an optimal anticipated residual astigmatism measurement, it can be seen at 158 that an Acriol-T10 IOL was chosen from chart 200, this IOL having a cylinder power of 6.00 diopters in the IOL plane and 4.2 diopters at the corneal plane, and an anticipated residual astigmatism of from emmetropic to 0.5 diopters corrected. As discussed herein, using this example, a low anticipated residual astigmatism may be calculated as discussed below, in accordance with embodiments of the invention.

Field 168 shows the pre-op corneal astigmatism which, as known in the art, may be calculated from the difference between the input Flat K measurement and the input Steep K measurement, shown in fields 126 and 130 (FIG. 5), respectively. In the example shown, field 168 was calculated according to the difference equation

47.00D@90°−41.00D@0°=6.00D×90°.

Field 172 shows the crossed-cylinder result (corneal plane) which, as known in the art, may be calculated from the difference between the pre-op corneal astigmatism and the surgically induced astigmatism at the corneal plane, the latter shown in both fields 136 (FIGS. 5) and 170 (FIG. 6). In the example shown, field 172 was calculated according to the difference equation

6.00D×90°−0.50D×0°=5.50D×90°.

In order to reduce the anticipated residual astigmatism, an IOL model was chosen from the chart 200 by choosing the model whose crossed-cylinder result (field 172) lies within the corresponding range of cylinder powers at the IOL plane and at the corneal plane. In the example shown, it was determined that the value 5.50, calculated above for field 172, lies within the range of 6.00 D to 4.2 D, which corresponds to the IOL model “Acriol-T10” in the chart 200. This IOL model is, therefore, the recommended IOL model for optimally reducing the resulting corneal astigmatism. This model is, therefore, output in field 158. Additionally, the cylinder power at the IOL plane and the cylinder power at the corneal plane, both shown in chart 200, are output in fields 164 and 166, respectively.

Finally, the anticipated residual astigmatism, shown in field 174, may be calculated from the difference between the calculated crossed cylinder result (corneal plane) and a cylinder power correction (corneal plane) of the toric IOL model. In the example shown, field 174 was calculated according to the equation

5.50×90°−4.20×0°=1.30×90°.

It may be noted that implanting a toric IOL in an eye with irregular astigmatism is likely to result in under correction and could also induce vision-degrading higher-order aberrations. Once this determination is made, the right spherical power of the IOL should be selected, using known methods.

As discussed herein, the present invention, in some embodiments thereof, provides a method and device which represent an improvement over known methods and devices. The present invention, in some embodiments thereof, provides a method and device for calculating the required power of a toric implant by using both the measured pre-operative corneal/ocular astigmatism and the predicted surgically-induced astigmatism. The surgically-induced astigmatism may be predicted by a surgeon by using known power vector analysis of the surgical technique to be employed during the IOL implantation procedure. The present invention, in some embodiments thereof, provides more accurate calculating of the required post-operative refractive power of the implant. The method is preferably automated by implementation on a computer through appropriate software.

It is expected that during the life of this patent many relevant toric IOLs will be developed and the scope of the term toric IOL is intended to include all such new technologies a priori.

As used herein the term “about” refers to ±10%.

Additional objects, advantages, and novel features of the present invention, in some embodiments thereof, will become apparent to one ordinarily skilled in the art upon examination of the following examples, which are not intended to be limiting. Additionally, each of the various embodiments and aspects of the present invention, in some embodiments thereof, as delineated hereinabove and as claimed in the claims section below finds experimental support in the following examples.

It is appreciated that certain features of some embodiments of the invention, which are, for clarity, described in the context of separate embodiments, may also be provided in combination in a single embodiment. Conversely, various features of some embodiments of the invention, which are, for brevity, described in the context of a single embodiment, may also be provided separately or in any suitable subcombination.

Although the invention, in some embodiments thereof, has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method for automatically calculating, prior to an IOL (intraocular lens) implantation procedure in a patient, a power and an axis location of the IOL in the implantation procedure, said method comprising:
inputting to an electronic device keratometry parameters of the patient;
inputting to the electronic device surgical parameters of the IOL implantation procedure on the patient, said surgical parameters including a surgically induced astigmatism (SIA) measurement;
analyzing the patient keratometry parameters and the surgical parameters by the electronic device;
determining a crossed-cylinder result (corneal plane), based on said analyzing, by the electronic device, wherein said crossed-cylinder result is calculated from the difference between a pre-op corneal astigmatism determined from the patient keratometry parameters and the SIA measurement;
comparing the determined crossed-cylinder result (corneal plane) with data in a database by the electronic device to select an IOL suitable for the implantation procedure, the selected IOL having cylinder power data corresponding to the crossed-cylinder result; and
after said selecting an IOL, automatically determining by the electronic device the power and the axis location of the selected IOL in response to said comparing.

2. A method according to claim 1, wherein the patient keratometry parameters include:
a Flat K measurement of the patient;
a Flat Axis degree measurement of the patient; and
a Steep K measurement and steep axis of the patient.

3. A method according to claim 2 wherein the Flat K measurement and the Steep K measurement may be input in one of diopters and millimeters.

4. A method according to claim 1, wherein the surgical parameters further include:
IOL spherical power; and
degree measurement of an incision location for the IOL implantation procedure.

5. A method according to claim 4 wherein each of the IOL spherical power and SIA measurement may be one of:
input in diopters, if the input Flat K and Steep K are input in diopters; and
input in millimeters, if the input Flat K and Steep K are input in millimeters.

6. A method according to claim 1, wherein said determining comprises determining the optimal power and axis location of the IOL in the implantation procedure.

7. A method according to claim 4, wherein the electronic device default for the SIA parameter is about 0.50 diopters.

8. A method according to claim 1, further comprising outputting the power and the axis location of the IOL in the implantation procedure by the electronic device.

9. A method according to claim 8, wherein said outputting comprises outputting the following:
at least one suggested toric IOL model for use in the IOL implantation procedure; and
an orientation axis for each of the at least one suggested toric IOL models.

10. A method according to claim 9, further comprising outputting at least one of:
a pre-op corneal astigmatism calculation;
a crossed-cylinder result (corneal plane) calculation; and
an anticipated residual astigmatism after the IOL implantation procedure, for each at least one suggested toric IOL model.

11. A method according to claim 9, wherein the at least one suggested toric IOL model includes two suggested toric IOL models.

12. A method according to claim 9, wherein said outputting includes outputting an eye diagram including an orientation for each of the at least one suggested toric IOL models.

13. A method according to claim 12, wherein said outputting includes providing an eye diagram with reference axis markings, the eye diagram indicating an incision location for and an axis location of the IOL in the implantation procedure.

14. A method according to claim 8, wherein said outputting is selected from displaying the power and the axis location of the IOL in the implantation procedure on a display and transmitting the power and the axis location of the IOL in the implantation procedure to a printing device.

15. A method according to claim 10, wherein said automatically determining further includes:
calculating the pre-op corneal astigmatism from the difference between an input Flat K measurement and an input Steep K measurement;
and
calculating the anticipated residual astigmatism from the difference between the calculated crossed cylinder result (corneal plane) and a cylinder power correction (corneal plane) of a suggested IOL model.

16. The method according to claim 9, wherein the suggested IOL model is acquired from a database of toric IOL data.

17. The method according to claim 15, wherein the cylinder power correction of the suggested IOL model is acquired from a database of toric IOL data.

18. A system for automatically calculating, prior to an IOL (intraocular lens) implantation procedure in a patient, the power and axis location of the IOL in the implantation procedure, said system comprising:
an input device configured for receiving keratometry parameters of the patient and surgical parameters of the IOL implantation procedure on the patient, the surgical parameters including a surgically induced astigmatism (SIA) measurement;
a processor configured for analyzing the patient keratometry parameters and the surgical parameters received from the input device and determining a crossed-cylinder result (corneal plane) wherein said crossed-cylinder result is calculated from the difference between a pre-op corneal astigmatism determined from the patient keratometry parameters and the SIA measurement;
a comparator configured for comparing the determined crossed-cylinder result (corneal plane) with data in a database to select an IOL suitable for the implantation procedure, the selected IOL having cylinder power data corresponding to the crossed-cylinder result and, after selecting the IOL suitable for the implantation procedure, automatically determining the power and axis location of the selected IOL in response to the comparing; and
a memory device configured for storing data.

19. A system according to claim 18, wherein the patient keratometry parameters include:
a Flat K measurement of the patient;
a Flat Axis degree measurement of the patient; and
a Steep K measurement of the patient.

20-34. (canceled)

35. A method according to claim 4, wherein the SIA measurement is selected from a range of from 0.0 D to 2.0 D.
